(19) United States
(12) Patent Application Publication
(10) Pub. No.: US 2020/0301998 A1
(43) Pub. Date: Sep. 24, 2020

(54) NOISE DETERMINATION METHOD, NOISE DETERMINATION DEVICE, AND NON-TRANSITORY COMPUTER-READABLE STORAGE MEDIUM FOR STORING NOISE DETERMINATION PROGRAM

(71) Applicant: FUJITSU LIMITED, Kawasaki-shi (JP)

(72) Inventors: Masatoshi Takenouchi, Hadano (JP); Tomoyuki Tsunoda, Kawasaki (JP); Yoshiaki Ikai, Fujisawa (JP); TAKASHI MIURA, Kawasaki (JP); Junji Kaneko, Mishima (JP); Takahiro Saito, Asaka (JP)

(73) Assignee: FUJITSU LIMITED, Kawasaki-shi (JP)

(21) Appl. No.: 16/819,289

(22) Filed: Mar. 16, 2020

(30) Foreign Application Priority Data
Mar. 22, 2019 (JP) 2019-055019

(51) Int. Cl. G06F 17/18; G16H 10/60; G16H 50/20

(52) U.S. Cl. CPC G06F 17/18; G16H 50/20; G16H 10/60

(57) ABSTRACT

A noise determination method executed by a computer, the noise determination method includes: acquiring time-series data; identifying a shape of a waveform of the time-series data using a persistent diagram; extracting a cluster whose lifetime from birth to death is equal to or greater than a threshold value in the persistent diagram; determining, from statistical information regarding time intervals related to pieces of data included in the cluster, whether or not peaks appear at regular intervals in the waveform of the time-series data; and controlling, based on a result of the determining, notification of an alert indicating that the time-series data includes noise.

### CROSS-REFERENCE TO RELATED APPLICATION

This application is based upon and claims the benefit of priority of the prior Japanese Patent Application No. 2019-55019, filed on Mar. 22, 2019, the entire contents of which are incorporated herein by reference.

### FIELD

The embodiments discussed herein are related to a noise determination method, a noise determination device, and a non-transitory computer-readable storage medium for storing a noise determination program.

### BACKGROUND

Electrocardiographic waves, brain waves, and signals emitted from the body due to biological phenomena such as pulse, respiration, and perspiration are analyzed to, for example, diagnose diseases or changes in physical conditions and find diseases at an early stage. For example, when brain waves are analyzed, noise may be mixed in brain wave data and decrease the accuracy. Examples of the noise include power-supply noise and noise generated by baseline fluctuations that occur when the contact states of electrodes or sensors are changed by body movement. Accordingly, a technique for removing noise from frequency data such as brain wave data using a frequency filter has been employed in recent years.

Examples of the related art include Japanese Laid-open Patent Publication No. 2019-16193, Japanese Laid-open Patent Publication No. 2011-110378, Japanese Laid-open Patent Publication No. 2004-249124, and Japanese Laid-open Patent Publication No. 2008-229307.

### SUMMARY

According to an aspect of the embodiments, a noise determination method executed by a computer, the noise determination method includes: acquiring time-series data; identifying a shape of a waveform of the time-series data using a persistent diagram; extracting a cluster whose lifetime from birth to death is equal to or greater than a threshold value in the persistent diagram; determining, from statistical information regarding time intervals related to pieces of data included in the cluster, whether or not peaks appear at regular intervals in the waveform of the time-series data; and controlling, based on a result of the determining, notification of an alert indicating that the time-series data includes noise.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram for describing a noise determination device according to a first embodiment;

FIGS. 2A to 2C illustrate feature extraction using TDA;

FIG. 3 is a diagram for describing brain waves;

FIG. 4 is a diagram for describing electrocardiographic waves;

FIG. 5 is a diagram for describing measurement data in which electrocardiographic waves are mixed in brain waves;

FIG. 6 is a diagram for describing an example of analysis using TDA;

FIG. 7 is a diagram for describing brain wave data having large amplitudes;

FIG. 8 is a functional block diagram illustrating a functional configuration of a noise determination device according to the first embodiment;

FIG. 9 is a diagram for describing how brain waves are measured;

FIG. 10 illustrates an analysis process;

FIG. 11 is a diagram for describing a result of analysis using statistical information;

FIG. 12 is a diagram for describing an example of a screen display;

FIG. 13 is a flowchart illustrating an overall flow of an analysis process;

FIG. 14 is a flowchart illustrating a detailed flow of a determination process;

FIG. 15 is a diagram for describing a determination process according to a second embodiment;

FIG. 16 is a diagram for describing a mixed pattern 1;

FIG. 17 is a diagram for describing a mixed pattern 2;

FIG. 18 is a diagram for describing a pattern where there is no mixture and only brain waves are included; and

FIG. 19 is a diagram for describing an example of a hardware configuration.

### DESCRIPTION OF EMBODIMENTS

With the technique described above, however, when the frequency band of the main component is the same between a target signal and noise, it is difficult to remove only noise. Even after filtering is performed on data, it is difficult to determine whether or not the data includes noise.

For example, assume that electrocardiographic waveform data is superimposed on brain wave data. In this case, the removal of the electrocardiographic waveform data using the frequency filter is difficult because both have the same frequency band as the main component. Therefore, the data may be unable to be used to diagnose a disease that would otherwise be derived from brain wave data. It may be possible for an expert to visually examine the brain wave data one by one to determine whether noise is mixed. However, this is not practical because it takes an enormous amount of time to process large amounts of patient data.

An object of an aspect of the present embodiments is to provide a noise determination method, a noise determination program, and a noise determination device that may improve noise mixture determination accuracy.

According to an embodiment, noise mixture determination accuracy may be improved.

Embodiments of a noise determination method, a noise determination program, and a noise determination device disclosed in the present application will be described in detail with reference to the drawings. The present disclosure is not limited to the embodiments. The embodiments may be appropriately combined as long as no contradiction occurs.

### First Embodiment

### Overall Configuration

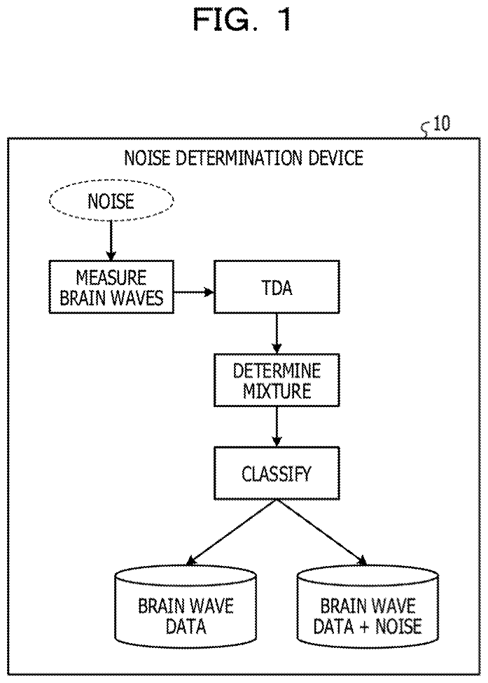

FIG. 1 is a diagram for describing a noise determination device according to a first embodiment. A noise determination device 10 illustrated in FIG. 1 is an example of a computer device that determines whether measured brain wave data includes noise and classifies measurement data based on the presence or absence of noise.

For example, as illustrated in FIG. 1, the noise determination device 10 extracts features of the shape of a waveform by using topological data analysis (TDA)-value-based filtration (VBF) on brain wave data measured by an electroencephalograph. The noise determination device 10 clusters the results of the extraction and determines whether noise is mixed based on the result of the clustering. Depending on the result of the mixture determination, the noise determination device 10 automatically classifies the data either into data that includes noise or data that does not include noise.

The analysis using TDA-VBF (hereinafter occasionally simply referred to as TDA) will be described. TDA-VBF is data analysis based on a topology called “topological data analysis” and characterizes the shape of data such as a figure and an image on a multiscale basis. For example, intersection points when a straight line, which is parallel to an axis of time-series data such as brain wave data, is moved are extracted as a topology. A persistent diagram is obtained from the extracted topology. In the persistent diagram, each point represents a chunk in the data. The features of the time-series data are extracted with one axis serving as a birth axis and another axis serving as a death axis. The birth axis represents birth parameters of chunks. The death axis represents death parameters of the chunks. For example, the time interval between the birth and the death of each chunk may be observed on the persistent diagram. The diagonal in the center of the diagram indicates that the time interval between the birth and the death of a chunk is zero. When the time interval between the birth and the death of a chunk is large, a diagram is generated in a position distant from the diagonal and the chunk may be regarded as noise. For example, in the case of electrocardiographic waves having a waveform with large amplitudes, the time interval from the birth to the death of a chunk is large. Accordingly, a diagram is generated in a position distant from the diagonal. In the case of brain waves with amplitudes that are smaller than amplitudes of the electrocardiographic waveform, the time interval from the birth to the death of a chunk is small. Accordingly, a diagram is generated in a position close to the diagonal.

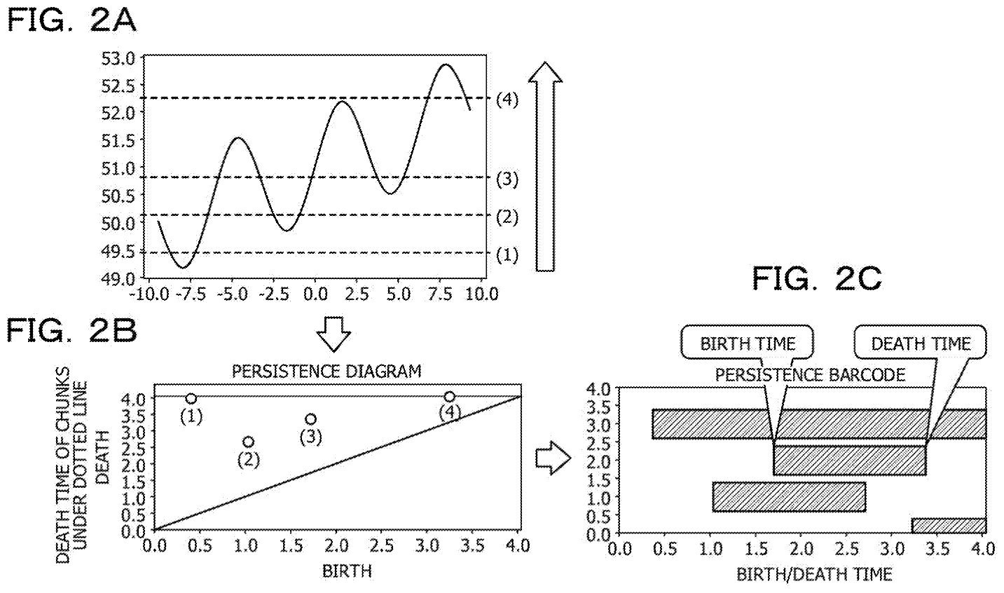

FIGS. 2A to 2C illustrate feature extraction using TDA. As illustrated in FIG. 2A, the measured brain wave data (hereinafter occasionally referred to as measurement data), which is data to be determined, is scanned from the bottom so that the timings of the birth and the death of the waves are extracted. For example, when the dotted line is moved from the bottom to the top of the measurement data, one chunk is formed below the dotted line at the timing of (1) in FIG. 2A. Another chunk is formed below the dotted line at the timing of (2) in FIG. 2A (two chunks in total). Still another chunk is formed below the dotted line at the timing of (3) in FIG. 2A (three chunks in total). Three chunks become one chunk below the dotted line at the timing of (4) in FIG. 2A.

As illustrated in FIG. 2B, a persistent diagram in which the birth (generation) time (birth) and the death time (death) of each chunk is plotted is generated and the lifetime of each chunk is extracted based on the distance from the diagonal where the lifetime is zero. After that, as illustrated in FIG. 2C, so-called barcode data is generated by plotting the lifetime of each chunk. From the barcode data, a Betti sequence indicating the feature values of the measurement data is generated and used for learning data, for example.

In the TDA analysis, analyzing the persistent diagram illustrated in FIG. 2B may be one possible technique to determine whether noise is mixed. However, this technique may have difficulty in determining whether noise is mixed.

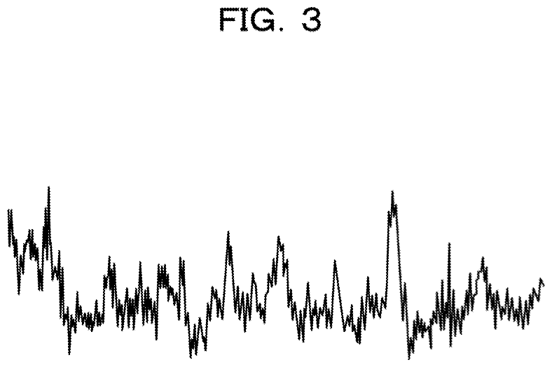

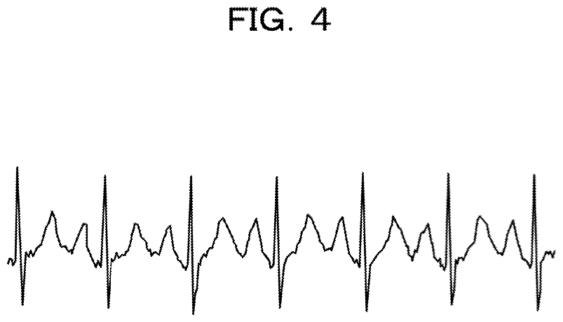

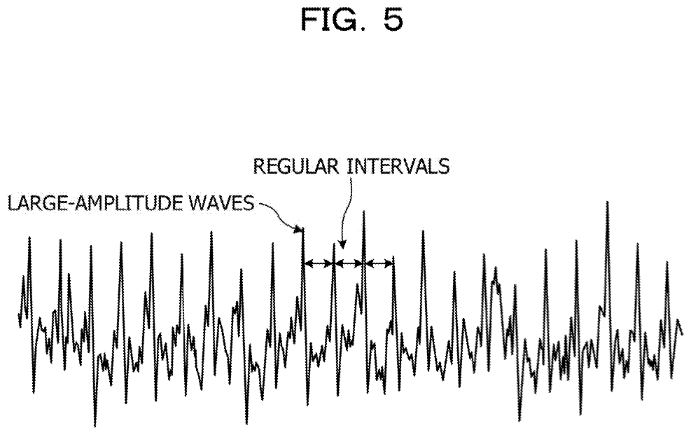

The waveforms assumed in the present application will be described below. FIG. 3 is a diagram for describing brain waves. FIG. 4 is a diagram for describing electrocardiographic waves. FIG. 5 is a diagram for describing measurement data in which electrocardiographic waves are mixed in brain waves. The brain waves illustrated in FIG. 3 have such features that the frequency range is 0.5 to 30 Hz, the waveform amplitude is 20 to 70 μV, and there is no periodicity. The electrocardiographic waves illustrated in FIG. 4 have such features that the frequency range is 0.05 to 100 Hz, the waveform amplitude is approximately 300 μV, and there is periodicity. In many cases, electrocardiographic waves have higher peaks than brain waves, and these peaks appear regularly. Therefore, as illustrated in FIG. 5, when the electrocardiographic waves are mixed in the brain waves, waves with large amplitudes may be detected at regular intervals, as compared to when only the brain waves are included.

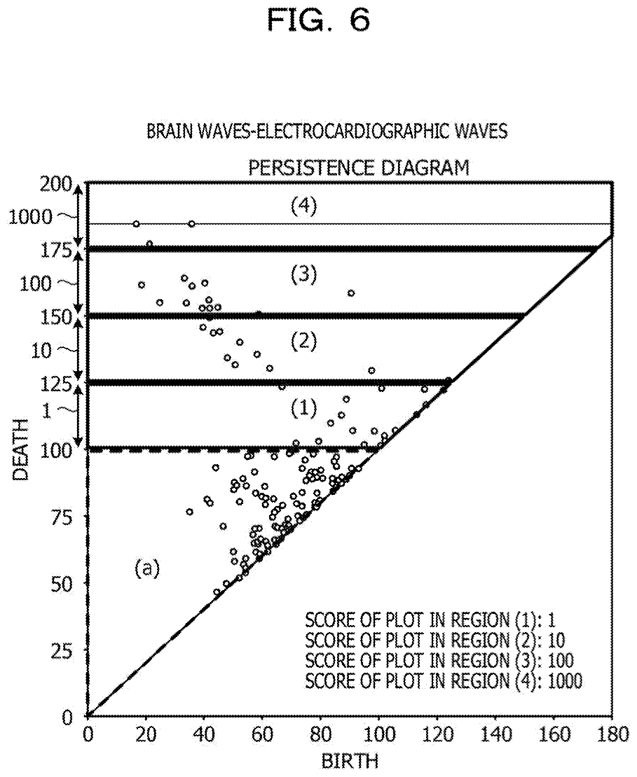

On the assumption that each waveform has the features described above, analysis of noise mixture using TDA will be described. FIG. 6 is a diagram for describing an example of analysis using TDA. As illustrated in FIG. 6, brain wave data (measurement data) for a certain period of time, which is data to be determined, is subject to TDA and then plotted in a persistent diagram. After the plot result is divided into regions and scores are set for individual regions, the scores of the measurement data, which is data to be determined, are totaled. Then, it is determined, according to the score total, whether or not noise is mixed in the measurement data.

For example, a region (a) in FIG. 6 includes chunks that do not have large amplitudes and have relatively short lifetimes. Therefore, the region (a) may be determined as data that is highly likely to correspond to brain waves, and is excluded when scores are totaled for noise mixture determination. Meanwhile, the electrocardiographic waves have large amplitudes as illustrated in FIG. 5. Accordingly, the electrocardiographic waves are highly likely to be plotted in positions distant from the diagonal. For each of regions (1) to (4), therefore, the score is set such that the value increases as the distance from the diagonal increases, for example, as the lifetime increases. It is assumed that 1 is set to the region (1), 10 is set to the region (2), 100 is set to the region (3), and 1000 is set to the region (4).

Under these conditions, there assume to be 17 pieces of data belonging to the region (1), 10 pieces of data belonging to the region (2), 12 pieces of data belonging to the region (3), and three pieces of data belonging to the region (4). In this case, the score is calculated as “(1×17+10×10+100×12+1000×3)=4317.” When the score is equal to or greater than a threshold value, it is determined that noise such as electrocardiographic waves is highly likely to be mixed. Therefore, the data is excluded from disease diagnosis data.

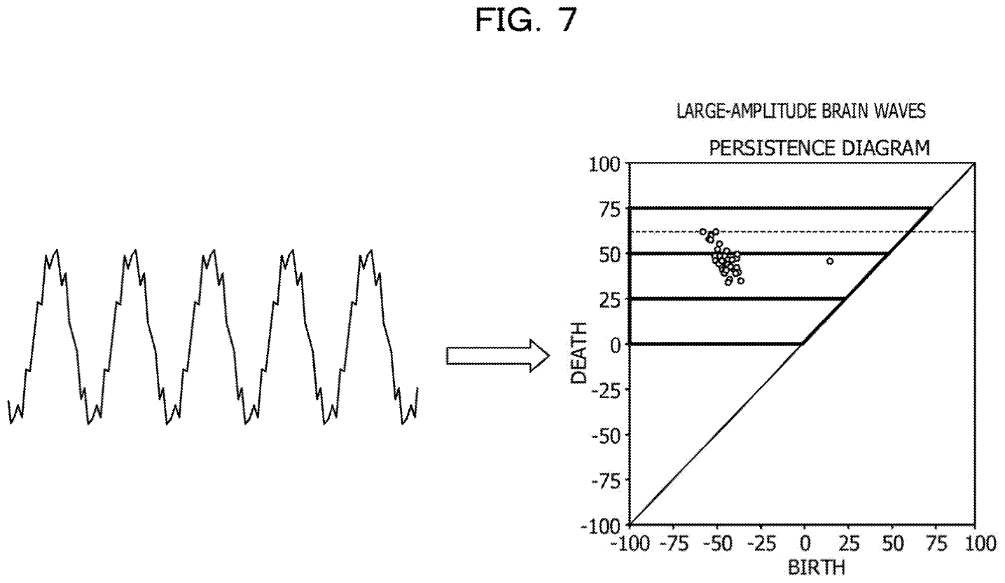

In some cases, however, even brain waves alone may have large amplitudes depending on the surrounding environment, human brain activation, and how devices are installed. FIG. 7 is a diagram for describing brain wave data having large amplitudes. In some cases, as illustrated in FIG. 7, while brain wave data is normal, large amplitudes may be measured due to factors other than noise. When such brain wave data having large amplitudes is analyzed using a persistent diagram, pieces of data are concentrated in positions distant from the diagonal.

In the case of the brain wave data having large amplitudes illustrated in FIG. 7, for example, many pieces of data appear in the region (3) and the region (4) illustrated in FIG. 6. This results in a large score value. Therefore, when the brain wave data is normal but the amplitudes are large, the brain wave data is determined to include noise under the technique using general TDA where the feature values are scored depending on the regions. This results in a decrease in noise mixture determination accuracy.

In view of the foregoing, in the present embodiment, when large-amplitude waves continue at certain regular intervals for a long period of time, it is determined that the waves are not normal brain waves and electrocardiographic waves are mixed therein. This improves noise mixture determination accuracy.

[Functional Configuration]

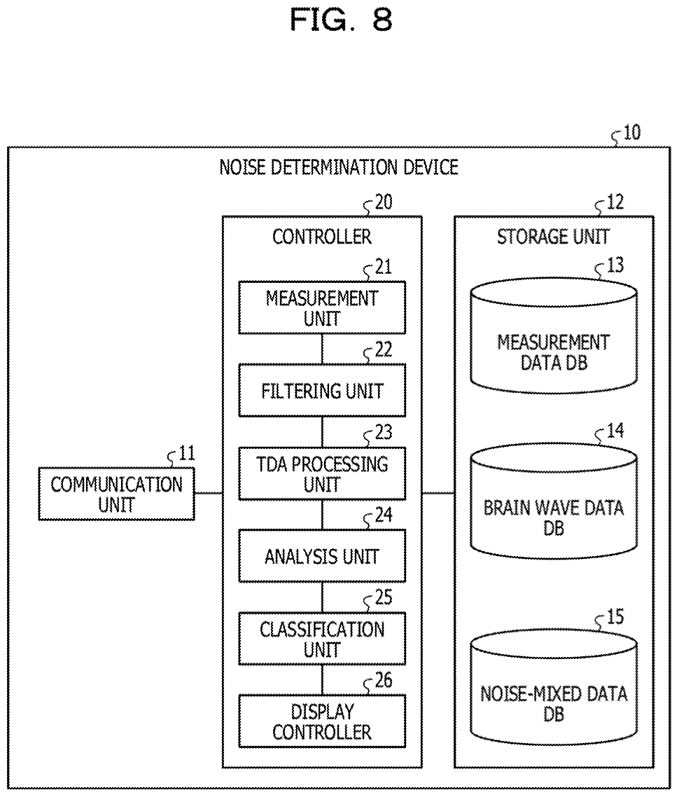

FIG. 8 is a functional block diagram illustrating a functional configuration of a noise determination device according to the first embodiment. As illustrated in FIG. 8, the noise determination device 10 includes a communication unit 11, a storage unit 12, and a controller 20.

The communication unit 11 is a processing unit that controls communication with other devices. The communication unit 11 is, for example, a communication interface or the like. For example, the communication unit 11 receives brain wave data (measurement data), which is data to be determined, from an electroencephalograph, and transmits a result of determination and the like to an administrator terminal.

The storage unit 12 is an example of a storage device that stores data, a program to be executed by the controller 20, and the like. The storage unit 12 is, for example, a memory, a hard disk, or the like. The storage unit 12 stores a measurement data database (DB) 13, a brain wave data DB 14, and a noise-mixed data DB 15.

The measurement data DB 13 is a database that stores measurement data. The measurement data is brain wave data received from the electroencephalograph and subject to noise mixture determination. For example, the measurement data DB 13 stores data to be determined, which has been measured as brain wave data and is unknown about whether noise is mixed therein.

The brain wave data DB 14 is a database that stores data that has been determined that no noise is mixed therein or the degree of noise mixture is within an allowable range. For example, the brain wave data DB 14 stores data that has been determined to be brain wave data by the controller 20 described later and that is usable as disease diagnosis data.

The noise-mixed data DB 15 is a database that stores data that has been determined that noise is mixed therein or the degree of noise mixture is out of the allowable range. For example, the noise-mixed data DB 15 stores data that has been determined to be brain wave data including many noises by the controller 20 described later and that may possibly hamper accurate diagnosis since the data is not suitable to be used as disease diagnosis data.

The controller 20 is a processing unit that controls the processes of the entire noise determination device 10. The controller 20 is, for example, a processor or the like. The controller 20 includes a measurement unit 21, a filtering unit 22, a TDA processing unit 23, an analysis unit 24, a classification unit 25, and a display controller 26. The measurement unit 21, the filtering unit 22, the TDA processing unit 23, the analysis unit 24, the classification unit 25, and the display controller 26 are examples of processes that are executed by an electronic circuit included in the processor or the like, the processor, or the like.

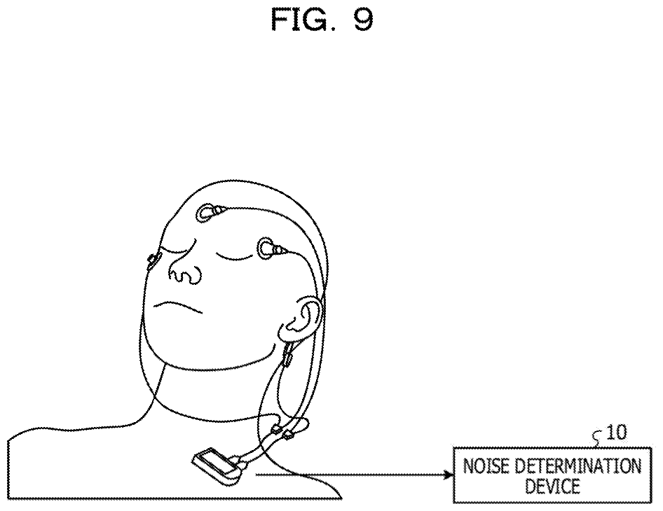

The measurement unit 21 is a processing unit that measures brain waves. For example, the measurement unit 21 acquires measured brain wave data from the electroencephalograph that measures brain waves, and stores the brain wave data in the measurement data DB 13 as measurement data. FIG. 9 is a diagram for describing how brain waves are measured. As illustrated in FIG. 9, the electroencephalograph measures brain waves through sensors attached to the head and transmits brain wave data, which is data of the measured brain waves. Upon measurement, the electroencephalograph is often placed in contact with or in the vicinity of the body of the subject to be measured. This may result in mixture of noise such as electrocardiographic waves.

The filtering unit 22 is a processing unit that performs a filtering process on measurement data, which is data to be determined. For example, the filtering unit 22 reads the measurement data from the measurement data DB 13 and applies a frequency filter to the measurement data to remove a frequency band other than the brain waves. The filtering unit 22 then outputs the measurement data after the removal to the TDA processing unit 23.

The TDA processing unit 23 is a processing unit that analyzes the measurement data using TDA and generates a persistent diagram. For example, the TDA processing unit 23 performs feature extraction using TDA, which has been described with reference to FIGS. 2A and 2B, on the measurement data input by the filtering unit 22 to generate the persistent diagram illustrated in FIG. 2B or FIG. 6. In this manner, the features of the shape of the waveform of the measurement data are represented by the diagram. The TDA processing unit 23 then outputs the persistent diagram corresponding to the measurement data to the analysis unit 24.

The analysis unit 24 is a processing unit that analyzes the persistent diagram corresponding to the measurement data generated by the TDA processing unit 23 and determines whether noise is mixed. For example, the analysis unit 24 clusters pieces of data plotted in the persistent diagram. The analysis unit 24 generates “distant cluster” and “close cluster.” “Distant cluster” is in a distance equal to or greater than a threshold value from the diagonal. “Close cluster” is in a distance less than the threshold value from the diagonal. When no “distant cluster” is present or when the number of samples (pieces of data) belonging to “distant cluster” is less than a threshold value, the analysis unit 24 determines that no noise is mixed, and outputs the result of the determination to the classification unit 25 and the display controller 26.

When “distant cluster” is present or when the number of samples (pieces of data) belonging to “distant cluster” is equal to or greater than the threshold value, the analysis unit 24 separates the distant cluster from each generated cluster. Subsequently, the analysis unit 24 extracts the peaks of large amplitudes of the separated cluster. After that, the analysis unit 24 detects the distribution of the time intervals between the peaks, and checks whether or not the peaks appear at certain regular intervals. When the large amplitudes continue to appear at regular intervals, the analysis unit 24 determines the measurement data as brain wave data in which noise is mixed.

For example, the analysis unit 24 determines whether noise is mixed by determining whether or not a long-lifetime data group, which corresponds to large amplitudes and appears in a position distant from the diagonal, has features of electrocardiographic waves whose large amplitudes appear at regular intervals. For example, when the data group has certain regularity, the analysis unit 24 gives an analysis that the large amplitudes appear at regular intervals, and determines that the measurement data includes electrocardiographic wave data. When the data group has no regularity, the analysis unit 24 gives an analysis that the brain wave data simply has large amplitudes, and determines that the measurement data does not include electrocardiographic wave data. The analysis unit 24 then outputs the measurement data and the result of the analysis to the classification unit 25 and the display controller 26.

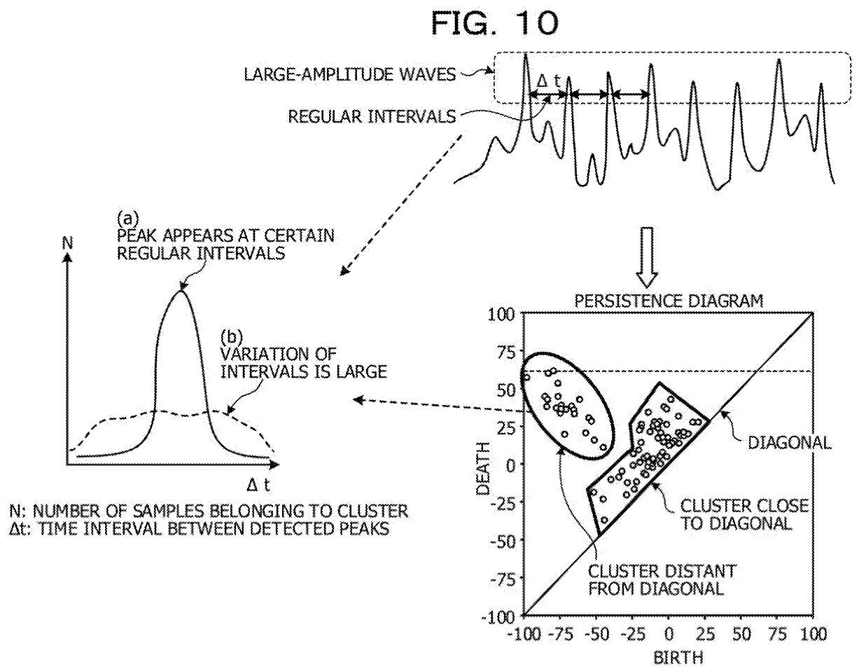

FIG. 10 illustrates an analysis process. As illustrated in FIG. 10, the analysis unit 24 clusters the plot results of the persistent diagram obtained from the measurement data into a cluster close to the diagonal and a cluster distant from the diagonal. While the analysis unit 24 may employ a general clustering technique, the analysis unit 24 classifies pieces of data that are in positions less than a given distance from the diagonal into one cluster (close cluster) and pieces of data that are in positions equal to or greater than the given distance from the diagonal into another cluster (distant cluster), for example. While two clusters are formed in FIG. 10, the number of clusters is not limited to two. When a large amplitude equal to or greater than a threshold value appears a plurality of times, a plurality of clusters distant from the diagonal may be formed.

The analysis unit 24 focuses on each “cluster distant from the diagonal” in a position equal to or greater than the threshold value from the diagonal. Subsequently, the analysis unit 24 refers to the measurement data to be analyzed, identifies large amplitudes (peaks) that are equal to or greater than the threshold value, and calculates the time intervals (Δt) between the peaks. After that, the analysis unit 24 identifies, for each time interval (Δt) between the peaks, the number of samples belonging to a corresponding one of the clusters that corresponds to the time interval between the peaks. Based on the identified number of samples, the analysis unit 24 determines whether peaks appear at regular intervals in the large-amplitude data group.

For example, as illustrated in FIG. 10, the analysis unit 24 selects, in order of appearance, each Δt identified from the original measurement data to be analyzed using TDA. Subsequently, the analysis unit 24 selects, in order of appearance, each cluster distant from the diagonal as the cluster corresponding to the selected Δt. In this manner, the analysis unit 24 associates each Δt with the cluster (distant cluster) corresponding to the Δt, and counts and graphs the number (N) of pieces of data belonging to each cluster.

When the plot result for each set of Δt and N has a shape with a peak as illustrated in (a) of FIG. 10, the analysis unit 24 determines that the peaks in the measurement data have regularity and the peaks appear at certain regular intervals. Therefore, the analysis unit 24 determines that noise is mixed in the measurement data. When the plot result for each set of Δt and N has a gradual shape without a peak as illustrated in (b) of FIG. 10, the analysis unit 24 determines that the peaks in the measurement data have no regularity. Therefore, the analysis unit 24 determines that no noise is mixed in the measurement data.

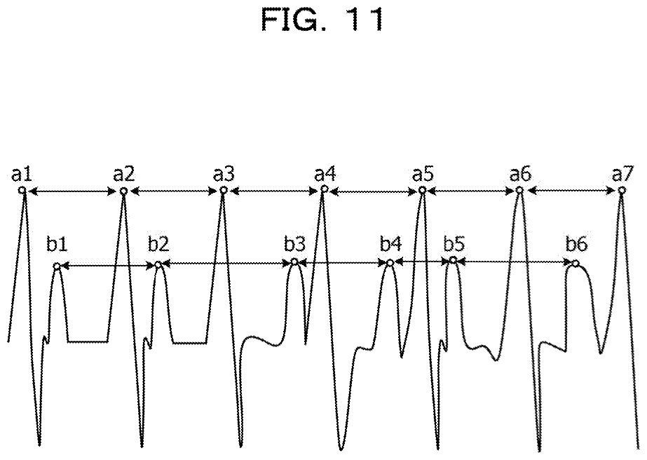

In order to increase the reliability of the result of analysis, the analysis unit 24 may determine whether or not peaks appear at certain regular intervals from statistical information of the measurement data itself. FIG. 11 is a diagram for describing a result of analysis using statistical information. As illustrated in FIG. 11, the analysis unit 24 converts the measurement data into coordinates and identifies, from the measurement data, a1 to a7 as peaks with amplitudes equal to or greater than a first threshold value. Based on the coordinates of each peak, the analysis unit 24 calculates each peak-to-peak distance. Then, the analysis unit 24 calculates the standard deviation of the peak-to-peak distances. When the standard deviation is less than a threshold value, the waveform interval is regular. Therefore, the analysis unit 24 determines that the peaks appear at regular intervals. When the standard deviation is equal to or greater than the threshold value, the waveform interval is not regular. Therefore, the analysis unit 24 determines that the peaks do not appear at regular intervals.

The analysis unit 24 may also identify, from the measurement data, b1 to b6 as peaks with amplitudes that are less than the first threshold value and equal to or greater than a second threshold value. For these peaks, the analysis unit 24 may calculate the standard deviation of the peak-to-peak distances and determine whether the peaks appear at regular intervals using the threshold value. Alternatively, when both the standard deviation of the peak-to-peak intervals from a1 to a7 and the standard deviation of the peak-to-peak intervals from b1 to b6 are less than the threshold value, the analysis unit 24 may determine that the peaks appear at regular intervals.

Referring back to FIG. 8, the classification unit 25 is a processing unit that classifies the measurement data based on the result of the analysis performed by the analysis unit 24. For example, the classification unit 25 stores, in the brain wave data DB 14, the measurement data determined by the analysis unit 24 that no noise is mixed therein. In the brain wave data DB 14, the classification unit 25 also stores the measurement data determined by the analysis unit 24 that peaks appear at irregular intervals rather than certain regular intervals. In the noise-mixed data DB 15, the classification unit 25 stores the measurement data determined by the analysis unit 24 that peaks have regularity and the peaks appear at certain regular intervals.

The display controller 26 is a processing unit that displays the result of the classification. For example, the display controller 26 displays the result of the classification performed by the classification unit 25 and the result of the analysis performed by the analysis unit 24 on a display unit such as a display and/or transmits the results to the administrator terminal or the like.

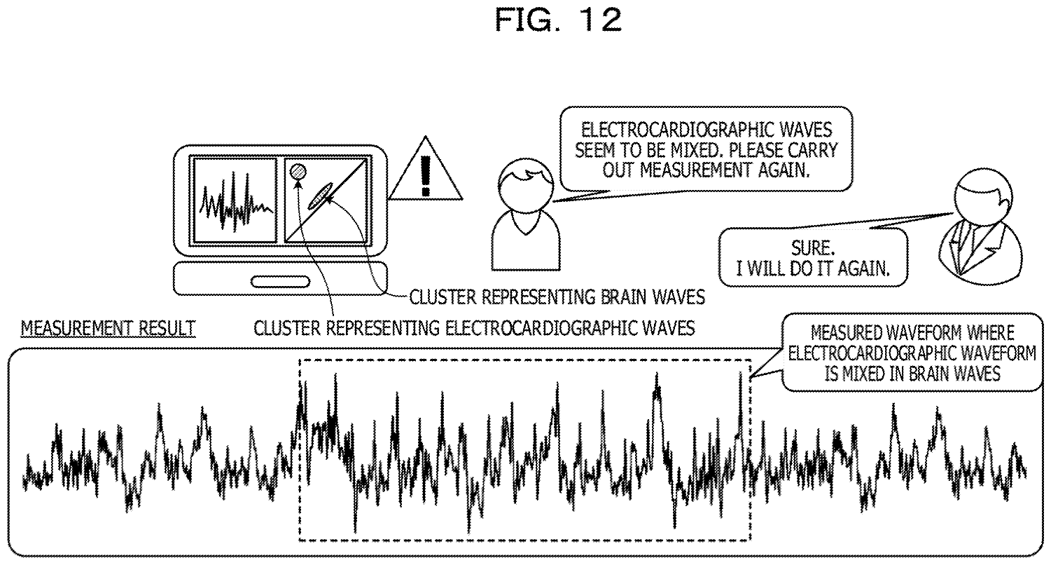

For example, assume that brain wave data has been measured to diagnose a disease in a medical institution. FIG. 12 is a diagram for describing an example of a screen display. As illustrated in FIG. 12, the display controller 26 displays both brain wave data measured by a medical professional and a persistent diagram that is the result of the analysis of the brain wave data on a computer, and notifies a doctor of whether noise is mixed. When noise is mixed in the brain wave data, the display controller 26 may also display a message or the like for prompting remeasurement.

The display controller 26 may also identify a position in which noise is mixed from the result of the analysis of the data of the series of measured brain waves and highlight the position on the brain wave data, for example. In this manner, the display controller 26 may also notify the doctor of the position that is not suitable for diagnosis.

[Flow of Analysis Process]

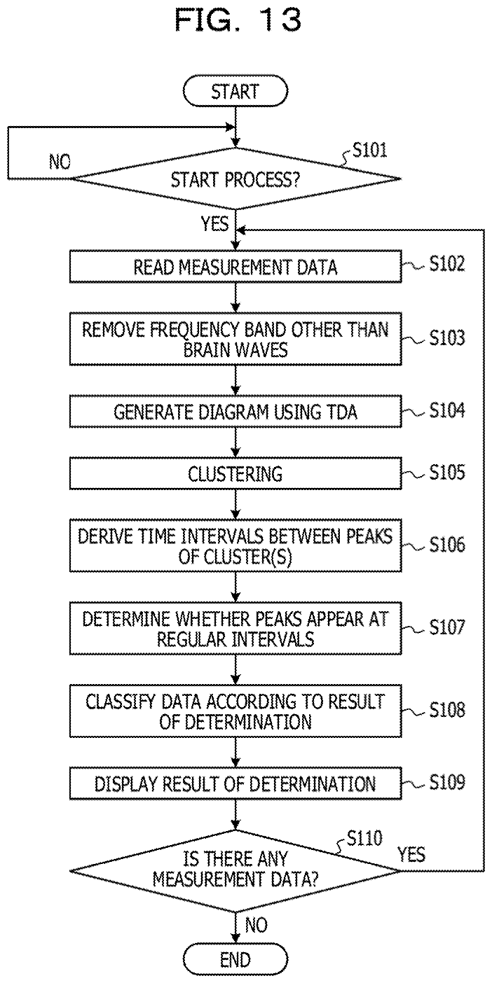

A flow of the above-described process of analyzing whether or not noise is mixed in measurement data will be described. FIG. 13 is a flowchart illustrating an overall flow of an analysis process. As illustrated in FIG. 13, after instruction from the administrator terminal and/or completion of the measurement of brain wave data, the measurement data is stored by the measurement unit 21 and a process start instruction is issued (S101: Yes). Subsequently, the filtering unit 22 reads the measurement data from the measurement data DB 13 (S102).

The filtering unit 22 applies the frequency filter to the measurement data to remove the frequency band other than brain waves (S103). The TDA processing unit 23 performs a TDA process on the measurement data after the removal, and generates a persistent diagram (S104).

After that, the analysis unit 24 clusters the results of the persistent diagram (S105), and derives the time intervals between peaks of cluster(s) distant from the diagonal of the diagram (S106). Subsequently, the analysis unit 24 determines whether or not the peaks appear at regular intervals (S107).

The classification unit 25 classifies the measurement data according to the result of the determination performed by the analysis unit 24 (S108), and the display controller 26 displays the result of the determination on the display or the like (S109). When there is any other measurement data to be analyzed (S110: Yes), processes in and after S102 are repeated. When there is no measurement data to be analyzed (S110: No), the process ends.

(Flow of Determination Process)

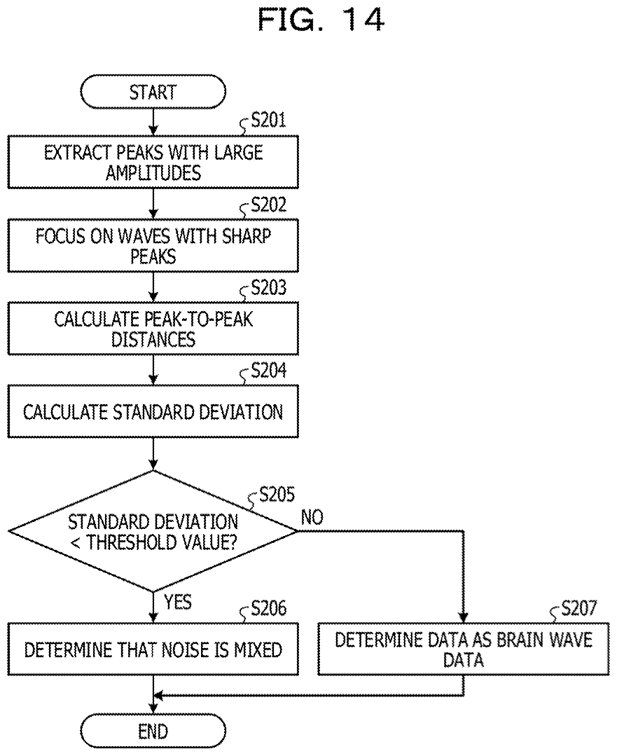

A flow of the determination process using the technique described with reference to FIG. 11 will be described. FIG. 14 is a flowchart illustrating a detailed flow of a determination process. For example, this process is performed in S107 of FIG. 13.

As illustrated in FIG. 14, the analysis unit 24 extracts peaks with large amplitudes (S201), and focuses on waves with sharp peaks (S202). Subsequently, the analysis unit 24 calculates the peak-to-peak distances (S203), and calculates the standard deviation of the peak-to-peak distances (S204).

When the standard deviation is less than the threshold value (S205: Yes), the analysis unit 24 deduces that the waveform has peaks at regular intervals, and determines that noise is mixed in the measurement data (S206). When the standard deviation is equal to or greater than the threshold value (S205: No), the analysis unit 24 deduces that the peak-to-peak intervals are irregular, and determines that no noise is mixed in the measurement data (S207).

[Effects]

As described above, when the measured brain wave data (measurement data) includes large-amplitude waves that occur at certain regular intervals for a long period of time, the noise determination device 10 determines that these waves are not normal brain waves and electrocardiographic waves are mixed therein. The noise determination device 10 may automate determination by calculating scores based on the brain wave data and the data in which electrocardiographic waves are mixed in brain waves and comparing the score of the brain wave data with the score of the data in which the electrocardiographic waves are mixed in the brain waves. Accordingly, an artifact of an electrocardiogram may be detected from the time-series brain wave information without individually recording the electrocardiogram.

In electrocardiographic waves having regular period and peak positions, the noise determination device 10 may easily grasp the features of a waveform of R waves or the like using TDA. As in the case of the electrocardiographic waves, moreover, the noise determination device 10 may easily perform feature extraction on brain waves having period and peaks that irregularly change. By applying TDA to data in which electrocardiographic waves are mixed in brain waves, the features of the brain waves are distinguished from the features of the electrocardiographic waves. Therefore, whether noise is mixed may be sufficiently determined by visual observation.

### Second Embodiment

With the technique according to the first embodiment, even when waves are normal brain waves, there is a possibility that the waves are erroneously detected as noise when the amplitude of a specific frequency band such as α waves or β waves is large. In a second embodiment, in order to suppress such erroneous noise detection, whether the peak shape of a waveform is sharp is added to the electrocardiographic-wave mixture determination in the first embodiment.

For example, the analysis unit 24 separates a cluster close to the diagonal and a cluster distant from the diagonal from each other based on the results of the persistent diagram. The analysis unit 24 determines whether or not the standard deviation σ of the lifetimes of pieces of data included in the cluster close to the diagonal is equal to or greater than a given value by comparing the standard deviation σ with the hypotenuse of a right triangle in which the cluster distant from the diagonal serves as the vertex. For example, the standard deviation for the data including only brain waves is greater than the standard deviation for the data in which electrocardiographic waves are mixed. Therefore, when a ratio of the standard deviation σ to the length of the hypotenuse of the right triangle is equal to or greater than a threshold value, the analysis unit 24 determines that the data includes only brain waves.

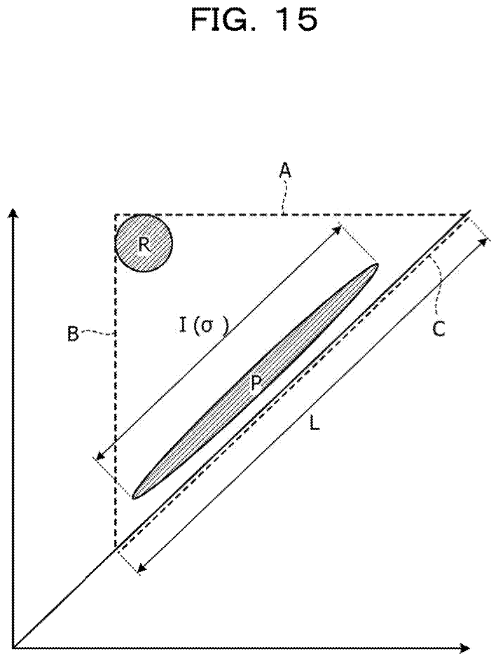

FIG. 15 is a diagram for describing a determination process according to the second embodiment. As illustrated in FIG. 15, the analysis unit 24 separates a cluster P and a cluster R from each other based on the results of the persistent diagram. The cluster P is close to the diagonal. The cluster R is distant from the diagonal. Subsequently, the analysis unit 24 generates a right triangle having sides A, B, and C by drawing the sides A and B from the cluster R, which serves as the vertex of the right triangle, toward the diagonal, which serves as the hypotenuse of the right triangle. The analysis unit 24 calculates the ratio of the standard deviation σ of the cluster P to a length L of the side C of the right triangle. When the ratio is equal to or greater than the threshold value, the analysis unit 24 determines that no noise is mixed in the measurement data. While the standard deviation of the cluster P is used in the example described herein, the present embodiment is not limited thereto. A length l from end to end of the cluster P may also be used.

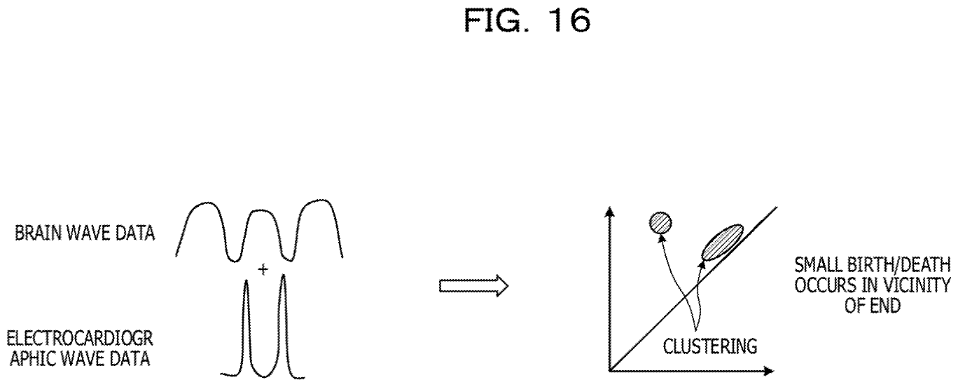

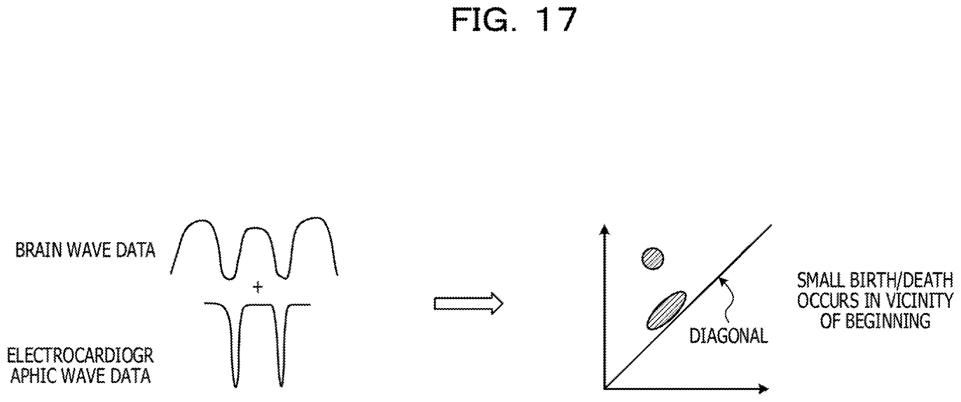

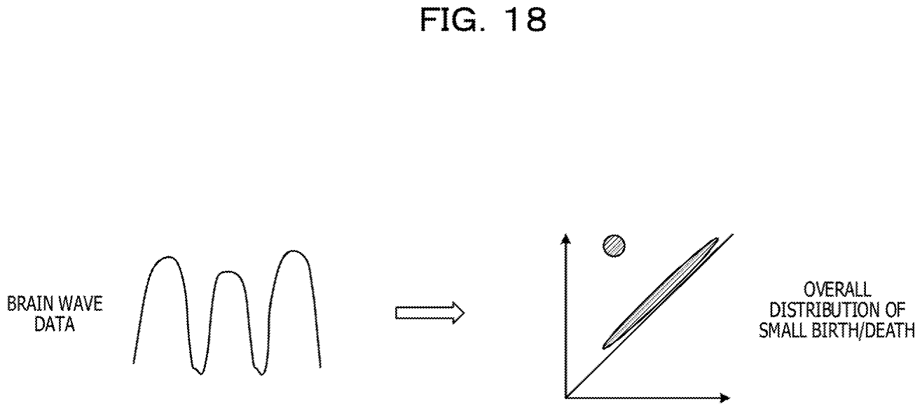

A mixed pattern where electrocardiographic waves are mixed and a pattern where only brain waves are included will be described. FIG. 16 is a diagram for describing a mixed pattern 1. FIG. 17 is a diagram for describing a mixed pattern 2. FIG. 18 is a diagram for describing a pattern where there is no mixture and only brain waves are included.

As illustrated in FIG. 16, a persistent diagram using TDA is generated from measurement data in which electrocardiographic wave data is mixed in brain wave data. The electrocardiographic wave data has large amplitudes and small peak widths. In this case, the birth and the death occur in the vicinity of the end of analysis using TDA. Accordingly, the cluster is generated in a rear portion of the diagonal (in the position distant from the origin). In the pattern illustrated in FIG. 16, therefore, the ratio of the standard deviation σ of the cluster P to the length L of the side C of the right triangle is small.

As illustrated in FIG. 17, a persistent diagram using TDA is generated from measurement data in which electrocardiographic wave data is mixed in brain wave data. The electrocardiographic wave data has large amplitudes and large peak widths. In this case, the birth and the death occur in the vicinity of the beginning of analysis using TDA. Accordingly, the cluster is generated in a front portion of the diagonal (in the position close to the origin). In the pattern illustrated in FIG. 17, therefore, the ratio of the standard deviation σ of the cluster P to the length L of the side C of the right triangle is small.

As illustrated in FIG. 18, a persistent diagram using TDA is generated from measurement data including only brain wave data. In this case, small birth and death occur across the analysis using TDA. In the pattern illustrated in FIG. 18, therefore, the ratio of the standard deviation σ of the cluster P to the length L of the side C of the right triangle is large.

In this manner, the standard deviation of the cluster close to the diagonal is compared with the hypotenuse of the right triangle in which the cluster distant from the diagonal serves as the vertex. This may suppress such a situation that normal brain wave data having large amplitudes in a specific frequency band is erroneously detected as noise.

### Third Embodiment

While the embodiments of the present disclosure have been described above, the present disclosure may be implemented in various different forms in addition to the embodiments above.

[Measurement Data]

While the brain wave data has been described as an example in the embodiments above, the measurement data is not limited thereto and other time-series data having irregular peaks may also be similarly processed.

[Analysis]

While both the analysis illustrated in FIG. 10 and the analysis using the statistical information illustrated in FIG. 11 are performed in the example in the embodiments above, the way the analysis is performed is not limited thereto and one of these analysis processes may be performed to determine whether noise is mixed. In this case, since the number of processes to be performed is reduced, the determination process may speed up. Moreover, the analysis using the statistical information illustrated in FIG. 11 is not limited to using the standard deviation and may use an average value or the like.

While the standard deviation or the length of the cluster close to the diagonal is compared with the hypotenuse of the right triangle in the example in the second embodiment, the way the determination is made is not limited thereto. For example, it may be determined whether or not the standard deviation or the length of the cluster close to the diagonal is equal to or greater than a threshold value. In this case, when the standard deviation or the length of the cluster close to the diagonal is equal to or greater than the threshold value, the possibility of noise mixture may be determined to be low.

[Noise]

While electrocardiographic waves have been described as an example of noise that is mixed in brain waves in the embodiments above, noise is not limited thereto and pulse waves or the like may be similarly processed. Moreover, while the output of the message for prompting remeasurement has been described as an example of an alert of noise mixture, the alert is not limited thereto and warning sound may be output or a warning lamp may light up, for example.

[System]

Unless otherwise specified, any change may be made to processing procedures, control procedures, specific names, and information including various pieces of data and parameters described in the embodiments above and drawings.

The components of the devices illustrated in the drawings are functional concepts and do not need to be physically configured as illustrated in the drawings. For example, concrete forms of the distribution and integration of the devices are not limited to those illustrated in the drawings, and all or part of the devices may be functionally or physically distributed or integrated in any desired unit depending on various loads and usage conditions.

All or any desired part of the processing functions to be executed by each device may be implemented by a central processing unit (CPU) and a program that is analyzed and executed by the CPU, or may be implemented as hardware using wired logic.

[Hardware]

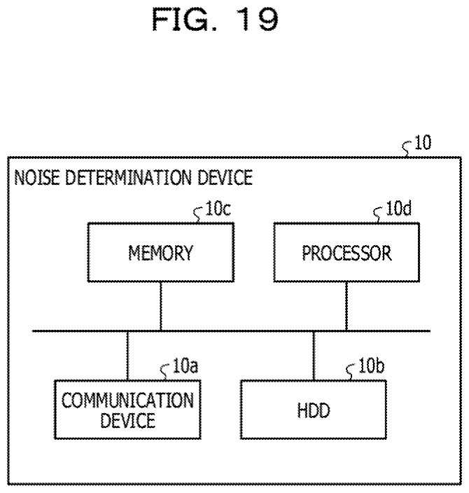

FIG. 19 is a diagram for describing an example of a hardware configuration. As illustrated in FIG. 19, the noise determination device 10 includes a communication device 10a, a hard disk drive (HDD) 10b, a memory 10c, and a processor 10d. The units illustrated in FIG. 19 are mutually coupled to each other via a bus or the like.

The communication device 10a is a network interface card or the like and communicates with other servers. The HDD 10b stores the program and the DBs that operate the functions illustrated in FIG. 8.

The processor 10d reads the program that executes the same processes as the processing units illustrated in FIG. 8 from the HDD 10b or the like, and loads the program into the memory 10c to operate the processes that execute the same functions as those described with reference to FIG. 8 and other figures. For example, these processes execute the same functions as the processing units included in the noise determination device 10. For example, the processor 10d reads the program having the same functions as the measurement unit 21, the filtering unit 22, the TDA processing unit 23, the analysis unit 24, the classification unit 25, the display controller 26, and the like from the HDD 10b or the like. Then, the processor 10d executes the processes that execute the same processes as the measurement unit 21, the filtering unit 22, the TDA processing unit 23, the analysis unit 24, the classification unit 25, the display controller 26, and the like.

In this manner, the noise determination device 10 reads and executes the program to operate as an information processing device that executes the noise determination method. The noise determination device 10 may cause a medium reader to read the program from a recording medium, and execute the read program to implement the same functions as those in the embodiments above. The program referred to herein is not limited to being executed by the noise determination device 10. For example, when another computer or server executes the program or when the computer and server execute the program in cooperation with each other, the present embodiments may also be similarly applied.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A noise determination method executed by a computer, the noise determination method comprising:
acquiring time-series data;
identifying a shape of a waveform of the time-series data using a persistent diagram;
extracting a cluster whose lifetime from birth to death is equal to or greater than a threshold value in the persistent diagram;
determining, from statistical information regarding time intervals related to pieces of data included in the cluster, whether or not peaks appear at regular intervals in the waveform of the time-series data; and
controlling, based on a result of the determining, notification of an alert indicating that the time-series data includes noise.

2. The noise determination method according to claim 1, wherein
the extracting includes extracting a plurality of clusters each having the lifetime equal to or greater than the threshold value, and
the determining includes
calculating peak-to-peak intervals that are intervals between peaks with amplitudes equal to or greater than a threshold value in the time-series data,
identifying, from the plurality of clusters, each individual cluster corresponding to pieces of data included in a corresponding one of the peak-to-peak intervals, and
determining that the time-series data includes the noise when a relationship between each peak-to-peak interval and the number of pieces of data included in the corresponding one of the peak-to-peak intervals is graphed and the graphed relationship takes a shape of a waveform with peaks.

3. The noise determination method according to claim 1, wherein
the determining includes
calculating a standard deviation of peak-to-peak intervals using intervals between peaks with amplitudes equal to or greater than a threshold value in the time-series data, and
determining that the time-series data includes the noise when the standard deviation is less than a threshold value.

4. The noise determination method according to claim 1, wherein
the identifying includes extracting a cluster having the lifetime less than the threshold value, and
the determining includes
calculating a standard deviation of lifetimes of respective pieces of data included in the cluster, and
determining, based on the standard deviation, whether or not the time-series data includes the noise.

5. The noise determination method according to claim 4, wherein
the determining includes
generating a right triangle in which a first cluster having the lifetime equal to or greater than the threshold value serves as a vertex of the right triangle and a diagonal of the persistent diagram serves as a hypotenuse of the right triangle,
calculating a ratio of a standard deviation of lifetimes of respective pieces of data included in a second cluster to a length of the hypotenuse of the right triangle, the second cluster having the lifetime less than the threshold value, and
determining that the time-series data includes the noise when the ratio is less than a threshold value.

6. A non-transitory computer-readable storage medium for storing a noise determination program which causes a processor to perform processing for object recognition, the processing comprising:
acquiring time-series data;
identifying a shape of a waveform of the time-series data using a persistent diagram;
extracting a cluster whose lifetime from birth to death is equal to or greater than a threshold value in the persistent diagram;
determining, from statistical information regarding time intervals related to pieces of data included in the cluster, whether or not peaks appear at regular intervals in the waveform of the time-series data; and
controlling, based on a result of the determining, notification of an alert indicating that the time-series data includes noise.

7. A noise determination device comprising:
a memory; and
a processor coupled to the memory, the processor being configured to
execute an acquisition processing that includes acquiring time-series data,
execute an identification processing that includes identifying a shape of a waveform of the time-series data using a persistent diagram,
execute an extraction processing that includes extracting a cluster whose lifetime from birth to death is equal to or greater than a threshold value in the persistent diagram,
execute a determination processing that includes determining, from statistical information regarding time intervals related to pieces of data included in the cluster, whether or not peaks appear at regular intervals in the waveform of the time-series data, and
execute a notification controlling processing that includes controlling, based on a result of the determining, notification of an alert indicating that the time-series data includes noise.
